(19) United States
(12) Patent Application Publication
(10) Pub. No.: US 2011/0293681 A1
(43) Pub. Date: Dec. 1, 2011

(54) HAND SANITIZING PATCH HAVING AN INTEGRALLY BONDED ANTIMICROBIAL

(75) Inventors: Judd Berlin, Naples, FL (US); Daniel Sigg, St. Paul, MN (US)

(73) Assignee: LecTec Corporation, Texarkana, TX (US)

(21) Appl. No.: 13/035,535

(22) Filed: Feb. 25, 2011

Related U.S. Application Data
(60) Provisional application No. 61/308,820, filed on Feb. 26, 2010.
(60) Provisional application No. 61/409,786, filed on Nov. 3, 2010.

(51) Int. Cl. A61K 9/70; A61K 31/7004; A01P 1/00; A61K 33/14; A61K 31/14; A61P 31/16; A61P 31/04; A61P 1/00; A61P 27/02; A61P 11/00; A61P 1/12; A61P 17/00; A61P 13/02; A61P 31/10; A61P 11/08; A61P 31/14; A61P 33/06; A61P 31/22; A61P 7/00; A61K 31/695

(52) U.S. Cl. 424/405; 514/63; 514/23; 424/664; 514/642

(57) ABSTRACT

The present invention provides for topical adhesive patch that includes a backing having a front side and a back side; a formulation in contact with the front side of the backing, the formulation including an adhesive; and at least one antimicrobial. The antimicrobial is integrally bonded to the back side of the backing. The present invention also provides for a method of reducing the number of microbes located upon a topical skin surface of a mammal, as well as a method of preventing the transmission of a communicable disease capable of being transmitted by physical contact. The methods include topically contacting a skin surface of the mammal with the back side of the topical adhesive patch.

### RELATED APPLICATIONS

This patent application claims the benefit of priority, under 35 U.S.C. §119(e), to U.S. Provisional Patent Application Ser. No. 61/308,820, filed on Feb. 26, 2010, and to U.S. Provisional Patent Application Ser. No. 61/409,786, filed on Nov. 3, 2010, both of which applications are incorporated herein by reference in their entirety.

### BACKGROUND OF THE INVENTION

Normal human skin is colonized with bacteria. The bacterial count of hands of medical personnel ranges from 3.9 to 4.6 million. Bacteria from the hands are divided into two categories: transient and resident. Transient flora is usually found on more superficial layers of the skin, and as such is more easily removed by routine hand washing. Transient flora is composed of organisms most often associated with health-care-associated infections. Resident flora is attached to the deeper layer of the skin and is more resistant to removal.

The primary function of the skin is to reduce water loss, provide protection against abrasive action and microorganisms, and act as a permeability barrier to the environment. The skin is divided in 4 layers, the superficial layer (horny layer or stratum corneum, 10 to 20 micrometers thick), the epidermis (50 to 100 micrometers), the dermis (1 to 2 mm), and the hypodermis (1 to 2 mm). The key barrier is in the stratum corneum. The region between cells in the stratum corneum is composed of lipid, and this lipid is necessary to form a competent skin barrier and forms a continuous domain.

There is substantial evidence that pathogens can be transmitted via hands, in particular in health-care settings (Guideline for Hand Hygiene in Health-Care Settings)—From Morbidity and Mortality Weekly Report, Oct. 25, 2002, 51, No. RR-16; Recommendations of the Healthcare Infection Control Practices Advisory Committee and the HICPAC, SHEA/APIC/IDSA Hand Hygiene Task Force (Center for Disease Control and Prevention.

Because, as noted above, many forms of infectious disease, such as the common cold, are contracted and spread to others, primarily by the hands, conventional medical wisdom teaches that the best way to prevent contraction and transmission of most common bacterial and viral infections is by thoroughly and often cleansing the hands throughout the day, and primarily before touching one's face or preparing food.

Cleansing and sanitizing an individual's hands using warm soapy water is generally effective for removing transient flora. For additional protection, antibacterial hand soaps may be utilized in lieu of conventional soap. Washing and rinsing of the hands is sometimes followed by applying a topical sterilizing agent such as, for example, a denatured alcohol, an anti-bacterial hand gel, or an anti-bacterial hand lotion to further protect the hands. Although these measures will help to control the spread of many pathogens residing on the skin, they are ineffective if not performed routinely and methodically. Unfortunately, routine use of antibacterial soaps, lotions, and gels may cause chapping of the skin or other undesirable reactions, especially for those persons who are sensitive to the active ingredients found in these products. Additionally, some individuals (e.g., children) may not have immediate access to facilities to adequately wash their hands with soap and water.

In the food service industry sanitation of hands is especially important for the protection of the public. Food service workers are therefore mandated by law to wash their hands after use of a restroom in order to prevent transmission of infectious disease, such as hepatitis, to food service customers. Because restrooms and wash facilities are generally private, regulations requiring hand washing of food service workers are nearly unenforceable and compliance therewith is usually only on an honor basis. However, concerns for public safety have become so great, possibly in light of more resistant strains of bacteria and viruses that have emerged in recent years, that additional safety measures have been proposed and implemented, including surveillance cameras in washrooms.

### SUMMARY OF THE INVENTION

The present invention provides for a topical adhesive patch. The patch includes a backing having a front side and a back side. At least one antimicrobial is integrally bonded to the back side of the backing. The patch includes a formulation that is in contact with the front side of the backing. The formulation includes an adhesive.

The present invention also provides for a method of reducing the number of microbes located upon a topical skin surface of a mammal. The method includes topically contacting a skin surface of the mammal with the back side of the topical adhesive patch described herein, effective to reduce the number of microbes located upon a topical skin surface.

The present invention also provides for a method of preventing the transmission of a communicable disease capable of being transmitted by physical contact. The method includes topically contacting a skin surface of the mammal at risk of such communicable disease, with the back side of the topical adhesive patch described herein, effective to prevent the transmission of the communicable disease.

Topically contacting the skin surface of the mammal with the back side of the topical adhesive patch can include, for example, rubbing the front side of a hand with the back side of the topical adhesive patch.

The antimicrobial is integrally bonded to the back side of the backing. In specific embodiments, upon use, little or no antimicrobial will be dispensed or transferred to the skin surface of the subject. In further specific embodiments, less than about 1 wt. %, less than about 0.5 wt. %, or less than about 0.1 wt. % of the antimicrobial from the topical adhesive patch is dispensed or transferred to the skin surface of the subject.

In specific embodiments, the topical adhesive patch mechanically and/or chemically inhibits, traps, removes, and/or kills harmful pathogens from the skin surface of the mammal. Additionally, in specific embodiments, the topical adhesive patch can remain active and effectively prevent the transmission of the disease for the requisite and suitable length of time, e.g., at least about 8 hours.

In specific embodiments, the communicable disease can include at least one of the following: common cold (rhinovirus), human Influenza (Influenzavirus A, Influenzavirus B, Influenzavirus C or Influenza H1N1), other respiratory infections (viral or bacterial), staphylococcus infections, streptococcus infections, gastroenteritis, bacterial meningitis, conjunctivitis, bacterial pneumonia, pertussis, tonsillitis, infectious diarrhea, cellulitis, impetigo, folliculitis, scalded skin syndrome, urinary tract infections, ringworm, athlete's foot, yeast infection, bronchiolitis, croup, measles, mumps, rubella, infectious diarrhea, encephalitis, conjunctivitis, chicken pox, west Nile virus, mononucleosis, cold sores, Avian influenza A (H5N1) virus, and other bacterial and viral infections.

In specific embodiments of the invention, the adhesive patch is relatively safe and non-toxic upon accidental ingestion. In additional specific embodiments of the invention, the adhesive patch causes minimal or no skin irritation. In additional specific embodiments of the invention, the adhesive patch causes little or no irritation upon contact with an eye, or surrounding tissue thereof.

### BRIEF DESCRIPTION OF THE DRAWINGS

FIG. 1 illustrates the front side of an adhesive patch of the present invention with a release liner attached to the patch.

FIG. 2 illustrates the back side of an adhesive patch of the present invention with a release liner attached to the patch, where the patch is partially detached from the release liner.

FIG. 3 illustrates one embodiment in which the adhesive patch of the present invention has an oval or elliptical shape.

FIG. 4 illustrates one embodiment in which the adhesive patch of the present invention covers a portion of the dorsal side of a hand.

FIG. 5 illustrates one embodiment in which the adhesive patch of the present invention covers the entire dorsal side of a hand.

FIG. 6 illustrates one embodiment in which the adhesive patch of the present invention covers the dorsal side of a hand from the wrist up to the fingernails (not including fingernails).

FIG. 7 illustrates an enlarged cross-sectional view of specific patch of the present invention.

FIG. 8 illustrates the diffusion of various components within an enlarged cross-sectional view of specific patch of the present invention.

FIG. 9 illustrates the patch manufacturing process of a specific patch of the present invention.

FIG. 10 illustrates a flow diagram summarizing the test procedures used in the study.

### DETAILED DESCRIPTION OF THE INVENTION

The present invention provides for a topical adhesive patch. The patch includes a backing having a front side and a back side. At least one antimicrobial is integrally bonded to the back side of the backing. The patch includes a formulation that is in contact with the front side of the backing. The formulation includes an adhesive.

Since the backing can be porous and/or vapor permeable, many consumers typically refer to the device as a “patch,” a “skin patch,” or an “adhesive skin patch.” As such, the device will herein be referred interchangeably to as a patch, a skin patch, and/or an adhesive skin patch. It is appreciated that those skilled in the art understand that the term “patch” is used to refer to the device and is not otherwise limiting in any manner.

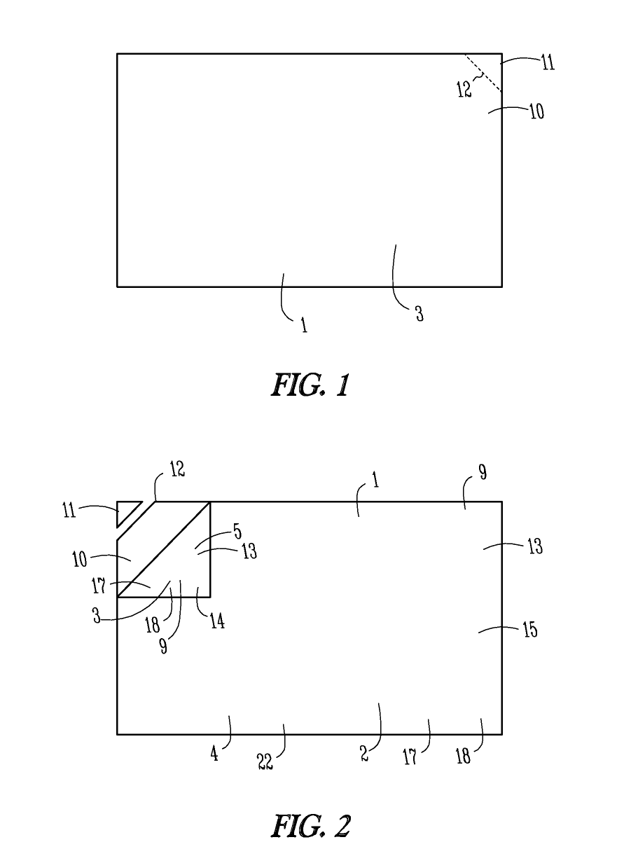

Referring to FIGS. 1-8, an exemplary adhesive patch 1 of the present invention is provided.

### Backing

The backing 2 is defined by a front side 3 (the side exposed to the subject, or an article of clothing of the subject, during use) and a back side 4 (the side exposed to the environment during use). The backing 2 should be nonirritating to human skin. The backing 2 is a self-supporting sheet of water soluble or water insoluble, polymeric or natural material that provides strength and integrity for the formulation 5. The backing 2 of the adhesive patch 1 can be vapor permeable. The backing 2 can also be porous, since porosity provides openings for receiving the formulation 5, and it helps to assure that the adhesive skin patch 1 is vapor permeable. Specifically, the backing 2 can retain the formulation 5 while allowing moisture from the skin to pass. Alternatively, the backing 2 can be non-porous. The backing 2 can have any suitable thickness. In specific embodiments, the suitable thickness allows for a flexible, bendable, pliable, vapor permeable, and/or a stretchable sheet of water insoluble porous material. Specifically, the thickness of the backing 2 can be about 0.001 mm to about 5.0 mm, about 0.001 mm to about 3.0 mm, or about 0.025 mm to about 1.25 mm.

The backing 2 can be manufactured from any suitable material. In specific embodiments, the suitable material forms a flexible, bendable, pliable, and/or stretchable backing 2. The backing 2 includes a porous or non-porous sheet of water soluble or water insoluble material that provides support for the adhesive skin patch 1. The backing 2 can include water soluble or water insoluble polymeric fibers, a porous film, or any other kind of matrix with spaces within the matrix. A specific backing 2 is a lightweight, porous, pliable strip composed of a nonwoven fabric of polymeric or natural fibers such as polyester, cotton, or cellulose fibers bonded together with a sizing resin. The backing 2 can be woven or nonwoven. In one embodiment, the backing 2 includes nonwoven fabric. Specifically, the backing 2 can include polyester fibers, polyurethane fibers, polyolefin fibers, polyamide fibers, natural fibers, cotton fibers, copolyester, copolyester fibers, cellulose acetate fibers, polycellulose fibers, or any mixture thereof. Additional stable, water insoluble flexible sheet materials and methods for manufacturing the suitable backings 2 are disclosed, e.g., in U.S. Pat. Nos. 4,675,009; 5,536,263; 4,696,854; 5,741,510; and references cited therein, and are suitable as backings 2 according to the present invention. The infusion of the formulation 5 into the backing 2 can be accomplished, e.g., with the use of a continuous process mixer, as disclosed, e.g., in U.S. Pat. No. 5,536,263, and references cited therein; or as discussed herein.

In some specific embodiments, the backing 2 can be composed of a micro-porous structure which will enable the patch 1 to mechanically trap and then also kill or inhibit germs (e.g. viruses, bacteria, fungi etc), and remove transient flora effective from the rubbing skin surface.

In some specific embodiments, the backing 2 comprises one or more integrally bonded binding substances comprising a human pathogen binding group for chemically binding a human pathogen to the backing binding substance. The binding group mechanically and/or chemically inhibits, traps, removes, and/or kills harmful pathogens. For example, a human pathogen binding group may be selected from the group consisting of a sulfate group (e.g. a sulfated monosaccharide or oligosaccharide) and/or a sulfonate group (e.g. a sulfonated monosaccharide or oligosaccharide) that can mimic the binding action of sialic acid groups of certain viruses such as adeno-associated virus (AAV), herpes simplex virus (HSV), influenza virus, and other human pathogens. The pathogen binding groups (e.g., the sulfate and/or sulfonate group) can be integrally bonded to free hydroxyl groups and/or free amino groups on fibers or fabrics of the backing. Alternatively, the pathogen binding groups can be integrally bonded to free hydroxyl groups and/or free amino groups on the fibers or fabrics of the backing using a sulfonate- or sulfate-containing dye that binds to the fabric. Backings of this type can be prepared cost-efficiently on an industrial scale. The backing 2 may further comprise one or more multivalent metallic ions or metallic salts that decrease the pathogenic capacity of one or more human pathogens, such as, for example, multivalent copper, multivalent silver, or multivalent zinc, all of which are viricidal, bactericidal, and fungicidal. In one embodiment, the metallic salt is a divalent metallic salt, such as copper oxide, zinc acetate, copper acetate, or copper sulfate, or mixtures thereof; all of which are bactericidal, viricidal, and fungicidal. Such materials and additional materials are described in U.S. Patent Publication 2010/0330140 the contents of which are incorporated herein by reference.

In a specific embodiment, the backing 2 can be manufactured from a substance or substances that are generally recognized as safe (GRAS) for topical use.

The backing 2 can be manufactured from a suitable non-woven fabric that is commercially available from, e.g., Freudenberg Faservliesstoffe K G (Weinham, Germany); Sontara Technologies (division of DuPont Corporation) (Old Hickory, Tenn.); Lystil S. A. (Brignoud Cedex, France); Dexter Nonwovens (Windsor Locks, Conn.); Testfabrics, Inc. (West Pittiston, Pa.); and Chicopee (New Brusnwick, N.J.). Other commercial vendors that supply suitable non-woven fabrics can be found at the Technical Textile website (http://www.technical-textiles.net/technical-textiles-index/orgL.htm).

Alternatively, the fibers of the backing 2 can be interlocked mechanically by air or water.

As shown in FIGS. 1-2 and 7-8, the backing 2 can include a front side 3 and a back side 4. The adhesive skin patch 1 can include a formulation 5 located in at least a portion of the front side 3 of the backing 2, on at least a portion of the front side 3 of the backing 2, or on and in at least a portion of the front side 3 of the backing 2. As such, the formulation 5 can be located on the entire surface of the front side 3 of the backing 2, or the formulation 5 can be located on a portion of the surface of the front side 3 of the backing 2.

In one embodiment, the formulation 5 can be located on the entire surface of the front side 3 of the backing 2. In addition to being located on the surface of the front side 3 of the backing 2, the formulation 5 can be located in at least a portion of the underlying surface of the front side 3 of the backing 2 (e.g., the formulation 5 can be partially embedded into the backing 2).

The formulation 5 can penetrate a substantial portion of the front side 3 of the backing 2, as disclosed, e.g., in U.S. Pat. No. 5,536,263, and references cited therein. For example, the formulation 5 can penetrate about one-tenth to about nine-tenths the thickness of the backing 2, or about one-fourth to about nine-tenths the thickness of the backing 2. As such, the formulation 5 can be partially embedded into the backing 2. In one embodiment, the formulation 5 can be located on the entire front side 3 of the backing 2 and partially in the front side 3 of the backing 2 (e.g., the formulation 5 is partially embedded into the backing 2).

Alternatively, a portion of the front side 3 of the backing 2 can include the formulation 5 and other portions of the front side 3 of the backing 2 can include any suitable and effective combination of the pressure sensitive adhesive 14 and, optionally, the solvent 13. For example, a central circular portion of the front side 3 of the backing 2 can include the formulation 5 while the remaining portions of the front side 3 of the backing 2 include only the pressure sensitive adhesive 14. The formulation 5, when partially embedded into the front side 3 of the backing 2, can impart strength and structure into the adhesive patch 1. For example, when the formulation 5 is partially embedded into the backing 2, the likelihood that the adhesive patch 1 tears apart when separated from the release liner 10 or when removed from the skin after use, is lowered.

When the adhesive skin patch 1 is placed upon the skin of a subject (e.g., a human), the formulation 5 can be in continuous contact with the skin surface of the subject.

In one embodiment, the adhesive skin patch 1, upon contact with skin, can allow the skin to breathe. In one embodiment, the adhesive skin patch 1, upon prolonged contact with skin, holds in place the formulation 5, and permits the skin to breathe over prolonged periods of time typically experienced with the use of the patch 1, e.g., up to about 7 days, up to about 24 hours, up to about 12 hours, up to about 8 hours, or up to about 6 hours.

As shown in FIG. 1, the adhesive skin patch 1 can be reversibly attached to a release liner 10. The release liner 10 helps to maintain the adhesiveness of the adhesive skin patch 1 prior to use, such as during manufacturing, packaging, shipping, and/or storage. Any suitable release liner 10 can be employed for use in the present invention. Suitable release liners 10 are readily known to those of skill in the art. See, e.g., U.S. Pat. Nos. 4,675,009; 5,536,263; 4,696,854; 5,741,510; and references cited therein for further descriptions of release liners 10 useful in the present invention. The release liner 10 can include a perforation 12 that allows the tab section 11 of the release liner 10 to be removed (see, FIGS. 1-2). Removal of the tab section 11 of the release liner 10 can allow the adhesive skin patch 1 to be removed from the release liner 10 with relative ease.

The backing 2 can be a porous or non-porous, self-supporting sheet of water insoluble or water soluble, polymeric or natural material that provides strength and integrity for the formulation 5. For example, the backing 2 can be water insoluble polymeric fibers, open cell foam backing (e.g., polyurethane, polyvinyl chloride, or polyethylene), a porous film, or any other kind of matrix with spaces within the matrix. In one embodiment, the backing 2 can include polyester, polyurethane, polyolefin, polyamide fibers, natural fibers, cotton fibers, polycellulose fibers, or any mixture thereof.

The back side 4 of the backing 2 of the patch 1 can be relatively dry to the touch, such that upon contact, e.g., with a skin surface or article of clothing, no appreciable or significant amount of liquid, gel, ointment, fluid, lotion, and the like, present in the back side 4 of the backing 2 of the patch 1 is transferred there from and deposited upon the skin surface or article of clothing.

The back side 4 of the backing 2 of the patch 1 can have a relatively small degree of moisture, while still being considered a “dry” patch, and would still be relatively dry to the touch, such that upon contact, e.g., with a skin surface or article of clothing, no appreciable or significant amount of liquid, gel, ointment, fluid, lotion, and the like, present in the back side 4 of the backing 2 of the patch 1 is transferred there from and deposited upon the skin surface or article of clothing.

The back side 4 of the backing 2 of the patch 1 can contact a skin surface through subsequent rubbing. In one embodiment, the subject can wear two adhesive patches 2, one on the dorsal aspect of each hand.

In one embodiment, the back side 4 of the backing 2 of the patch 1 can be relatively dry, upon touching.

### Antimicrobial

As used herein, the term “microbe” refers to both viruses and bacteria.

As used herein an “antimicrobial” refers to a substance that either traps, removes, kills, or inhibits the growth of a microorganism. Antimicrobials can be either antivirals or antibiotics. Typically, an antimicrobial kills a microorganism or inhibits their growth by cell wall damage, inhibition of cell wall synthesis, alteration of cell wall permeability, inhibition of the synthesis of proteins and nucleic acids, and inhibition of enzyme action.

In specific embodiments, selection of a suitable antimicrobial 15, that is integrally bonded to the backing 2, will provide a patch 1 that exhibits: effective control of bacteria, molds and fungi; selective activity towards undesirable microorganisms; absence of toxic effects for both the manufacturer and the subject user; durability of activity to leaching; applicability with no adverse effects on the fabric; acceptable moisture transport properties; compatibility with other finishing agents; and/or easy application, compatibility with common textile processing.

The patch 1 includes an antimicrobial 15, useful for preventing the transmission of a communicable disease capable of being transmitted by physical contact. The antimicrobial 15 is integrally bonded to the back side 4 of the backing 2. Any suitable antimicrobial 15 can be employed, provided the antimicrobial 15 can effectively kill microbes on the patch, and on contacting skin surface to ultimately prevent the transmission of a communicable disease capable of being transmitted by physical contact, and the antimicrobial 15 can be integrally bonded to the back side 4 of the backing 2. In one embodiment, the stability is over a prolonged period of time, e.g., up to about 2 years, up to about 1 year, or up to about 6 months, typically experienced in the manufacturing, packaging, shipping, and/or storage of the patch 1.

The antimicrobial 15 is integrally bonded to the back side 4 of the backing 2. In specific embodiments, less than about 5 wt. % of the antimicrobial 15 from the topical adhesive patch 1 is dispensed or transferred to the skin surface of the subject, upon use. In additional specific embodiments, less than about 1 wt. % of the antimicrobial 15 from the topical adhesive patch 1 is dispensed or transferred to the skin surface of the subject, upon use. In additional specific embodiments, less than about 0.5 wt. % of the antimicrobial 15 from the topical adhesive patch 1 is dispensed or transferred to the skin surface of the subject, upon use. In further specific embodiments, less than about 0.1 wt. % of the antimicrobial 15 from the topical adhesive patch 1 is dispensed or transferred to the skin surface of the subject, upon use. In further specific embodiments, about 0 wt. % of the antimicrobial 15 from the topical adhesive patch 1 is dispensed or transferred to the skin surface of the subject, upon use.

The antimicrobial 15 can be integrally bonded to the back side 4 of the backing 2 in any suitable manner. For example, the antimicrobial 15 can be integrally bonded to the back side 4 of the backing 2 with the use of at least one of heat, pressure, and radiant energy. It is appreciated that those of skill in the art understand, given a specific antimicrobial 15, how to effectively integrally bond the antimicrobial 15 to the back side 4 of the backing 2. Specifically, suitable methods of fabricating the patch 1 containing the antimicrobial 15 include, for example, addition, before extrusion, of the antimicrobial 15 to the material that forms the backing 2. Alternatively, the backing 2 can be treated with the antimicrobial 15 during the finishing stages of the patch 1. As stated above, suitable materials that form the backing 2 include, e.g., cotton fibers, cellulose fibers, polyester fibers, polyurethane fibers, polyolefin fibers, polyamide fibers, copolyester fibers, cellulose acetate fibers, polycellulose fibers, or any mixture thereof.

As used herein, “integrally bonded” refers to the relationship of the antimicrobial 15 and the backing 2. As used herein, “bonding” refers to attaching or combining the antimicrobial 15 and backing 2 by mechanical, chemical, electrostatic, friction, sequestration and/or complexing techniques, so that the antimicrobial 15 will not readily separate from the backing 2.

The antimicrobial 15 can be bonded to fibers of the backing 2, such that the antimicrobial is incorporated into the fibers when they are spun. Alternatively, the antimicrobial 15 can be bonded to surface of the back side 4 of the backing 2. The antimicrobial 15 can be bonded to a portion of the surface of the back side 4 of the backing 2, or can be bonded to the entire portion of the surface of the back side 4 of the backing 2. Additionally, the antimicrobial 15 can be chemically bonded to the backing 2. With chemical bonding, durability can be achieved employing suitable reactive groups on the textile (i.e., backing 2) and antimicrobial 15. The antimicrobial 15 can be chemically bonded to a portion of the back side 4 of the backing 2, or can be chemically bonded to the entire portion of the back side 4 of the backing 2.

In a specific embodiment, the nature and amount of the antimicrobial 15 is selected, such that it will be generally recognized as safe (GRAS) for topical use.

Suitable specific exemplary and non-limiting antimicrobials 15, classes of antimicrobials 15 and commercially available products incorporating the same are provided in the table below.

Integrally bonded antimicrobials

| Commercial | | |
|---|---|---|
| Product/ | General class of | |
| Technology | antimicrobial | Specific antimicrobial |
| AEGIS Microbe Shield ® SYLGARD ® Antimicrobial Treatment SiShield ® BIOSHIELD ® Bio Shield ® AM 500 | Silyl Quaternary Amine Quaternary ammonium cation (QACS) Organosilane Quatery Amine (SiQac) compound Silicon Quaternary Ammonium Salt Alkylammonium cation | 3-(Trimethoxysilyl)propyl dimethyl octadecyl ammonium chloride 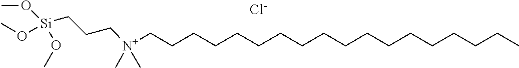 |
| Biosil ® | Silicic acid is a general name for a family of | Some simple silicic acids have been identified as metasilicic |
| | chemical compounds of the element silicon, | acid (H₂SiO₃), orthosilicic acid (H₄SiO₄), disilicic acid |
| | hydrogen, and oxygen, with the general | (H₂Si₂O₅), and pyrosilicic acid (H₆Si₂O₇); however in the solid |
| | formula [SiOₓ(OH)₄₋₂ₓ]ₙ. | state these probably condense to form polymeric silicic acids |
| | | of complex structure. |
| EcoFresh ® | Si-Quat based agents | Metallic and ionic silver (Ag+) |
| | (antimicrobial) | |
| Purista ® Reputex ™ | Polyaminopropyl Biguanide (PAPB) 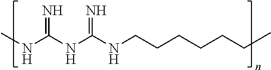 | Poly (hexamethylene biguanide) hydrochloride (PHMB) 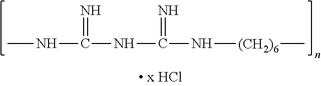 |
| Agiene ® | Silver Complex | Micro Silver Crystal |
| Agion ® | | |
| Coloplast | | |
| InterDry | | |
| Ag ® | | |
| BioFriend ™ | Polysaccharide N or O-substituted derivatives of neuraminic acid | Sialic Acid (N-acetyl-neuraminic acid, Neu5Ac, NAN, NANA) 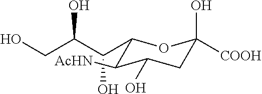 |
| Anson ® | Nano Silver particles | Wound healing is accelerated by silver nano-particles. |
| NIMBUS ® BIOGUARD ™ | Quaternary Ammonium Polymer Cationic Quaternary Polyelectrolyte | Poly(diallyldimethylammonium chloride) 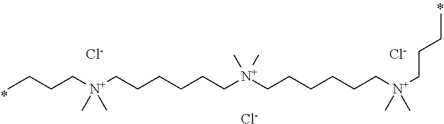 polyDADMAC and Poly (vinyl benzyltrimethyl ammonium chloride) (PVBTMAC) |
| Sanitized AG ® | Halogenated phenoxy- | 5-chloro-2-(2,4-dichloro-phenoxy)phenol |
| | compound and | or |
| | isothiazolinone derivate | 2,4,4′-trichloro-2′-hydroxy-diphenyl ether |
| | | 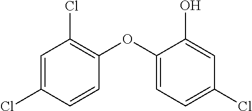 |
| | | Methylisothiazolinone |
| | | 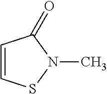 |
| Chitosan | Chito-oligosaccharides | [N-(2-hydroxyl)propy1-3-trimethylammonium chitosan |
| | | chloride] or HTCC |
| | | (a water-soluble chitosan quaternary ammonium derivative) |
| Peach Fresh ® | Tertiary Ammonium Compounds | Metallic silver, silver oxides, and silver salts. Nano Silver |
| | Silver complexes of poly(amidoamine) | |
| | (PAMAM) | |
| | Silver - PAMAM | |

The antimicrobial 15 can be employed in any suitable amount, provided the amount of antimicrobial 15 can effectively kill microbes on the patch 1, and on contacting skin surface to ultimately prevent the transmission of a communicable disease capable of being transmitted by physical contact, and the amount of antimicrobial 15 can be integrally bonded to the back side 4 of the backing 2.

The amount of antimicrobial 15 present in backing 2 typically depends upon the specific compound or compounds employed as the antimicrobial 15. For example, 3-(trimethoxysilyl)propyl dimethyl octadecyl ammonium chloride can be employed in up to about 10 wt. % of the patch 1, up to about 5 wt. % of the patch 1, or up to about 1 wt. % of the patch 1.

### Formulation

As shown in FIGS. 1-2 and 7-8, the backing 2 can include a front side 3 and a back side 4. The patch 1 can include a formulation 5 located in at least a portion of the front side 3 of the backing 2, located on at least a portion of the front side 3 of the backing 2, or located on and in at least a portion of the front side 3 of the backing 2. In one embodiment, the formulation 5 is located on the entire front side 3 of the backing 2 and partially in the front side 3 of the backing 2 (e.g., the formulation 5 is partially embedded into the backing 2).

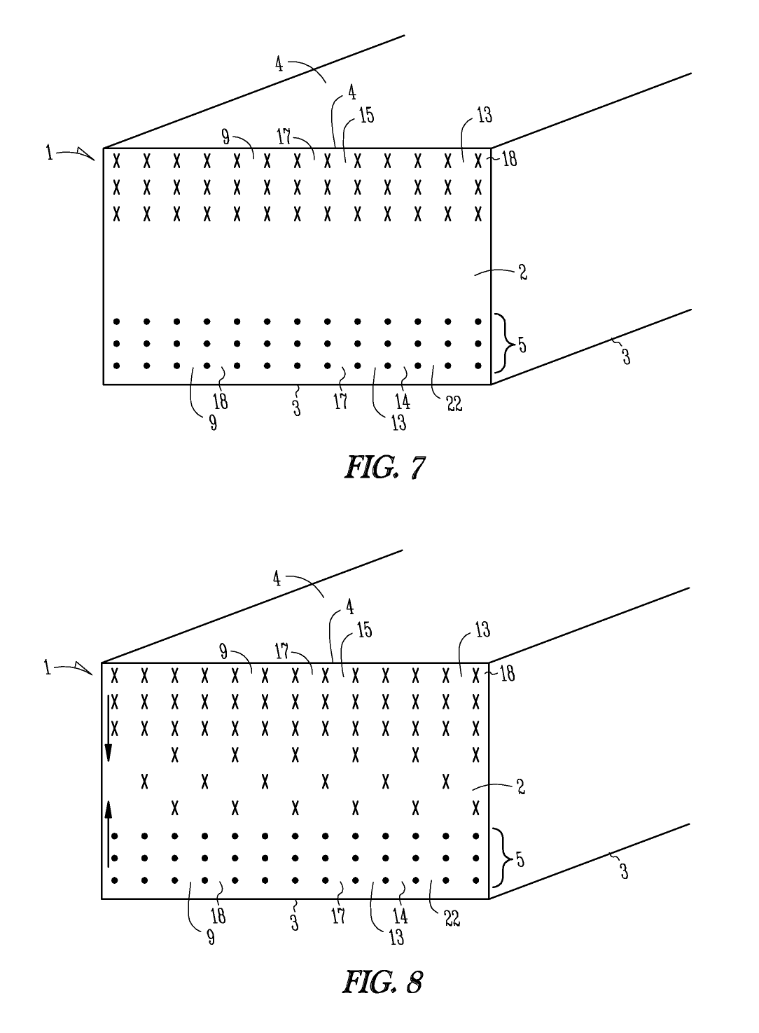

The formulation 5 can be positioned on and in any portion of the front side 3 of the backing 2, i.e., the formulation can be positioned on at least a portion on the front side of the backing, in at least a portion on the front side of the backing, or on and in at least a portion on the front side of the backing. The formulation 5 can be positioned in a portion of the front side 3 of the backing 2 (e.g., the formulation 5 penetrates a substantial portion of the front side 3 of the backing 2) as disclosed in, e.g., U.S. Pat. No. 5,536,263, and references cited therein. For example, the formulation 5 can penetrate a substantial portion of the front side 3 of the backing 2, e.g., typically between about one-fourth to about nine-tenths the thickness of the backing 2. The penetration of the formulation 5 into the backing 2 can be seen in FIGS. 7-8.

In one embodiment, the formulation 5 can be positioned on the entire front side 3 of the backing 2. In this latter configuration, the formulation 5 is in continuous contact with the entire front side 3 of the backing 2. When the adhesive skin patch 1 is placed upon the skin surface of a subject, the formulation 5 is in continuous contact with the skin surface of the subject.

Alternatively, a portion of the front side 3 of the backing 2 can contain the formulation 5 and other portions of the front side 3 of the backing 2 can contain any combination of the adhesive 14, and, optionally, the solvent 13. For example, a central circular portion of the front side 3 of the backing 2 can contain the formulation 5 while the remaining portions of the front side 3 of the backing 2 contains only the adhesive 14.

In a specific embodiment, the formulation 5 can be manufactured from a substance or substances that are generally recognized as safe (GRAS) for topical use.

The formulation 5 can include an adhesive 14 and, optionally, one or more of the following components: a solvent 13, one or more polymers 9, a humectant 17, a topical moisturizer 18, and one or more polyhydric alcohols 22.

The formulation 5 can remain stable over the period of time typically experienced with the manufacturing, packaging, shipping, and/or storage of the adhesive skin patch 1, e.g., up to about a month, up to about a year, or up to about two years.

### Solvent

The solvent 13 can act as a carrier for, and in one embodiment, can dissolve, the adhesive 14. Any suitable solvent 13 can be employed, provided the solvent 13 effectively and independently dissolves the adhesive 14, and the solvent 13 remains stable in the formulation 5. In one embodiment, the stability is over a prolonged period of time, e.g., up to about 2 years, up to about 1 year, or up to about 6 months, typically experienced in the manufacturing, packaging, shipping, and/or storage of the patch 1.

The solvent 13 can include one or more organic compounds, one or more inorganic compounds, or mixtures thereof. In one embodiment, the solvent 13 will include one or more organic compounds, e.g., esters, terpenes, alcohols, ketones, aldehydes, fatty acids, partially or fully esterified fatty acids, wherein the structures are cyclic, non cyclic (e.g., alkyl), alicyclic (e.g., a bridged ring compound) or aromatic, as well as organic compounds having combinations of these functional groups. Suitable exemplary solvents 13 are disclosed, e.g., in Aldrich Handbook of Fine Chemicals, 2000-2001 (Milwaukee, Wis.). In one embodiment, the solvent 13 includes water (e.g., deionized water).

In one embodiment of the present invention, the solvent 13 can include a (C₁-C₁₂) acyclic hydrocarbon, a (C₃-C₁₂) cyclic hydrocarbon, a (C₆-C₁₂) aryl hydrocarbon, a (C₆-C₁₂) heteroaryl hydrocarbon, or a (C₃-C₁₂) heterocyclic hydrocarbon;

wherein any of the hydrocarbons can optionally include one or more carbon-carbon double bonds and any of the hydrocarbons can optionally include one or more carbon-carbon triple bonds;

wherein any of the hydrocarbons can optionally include one or more oxy (—O—), carbonyl (—C(═O)C—), carboxylato (—C(═O)O—), dioxy (—O—O—), dithio (—S—S—), imino (—NH—), methylene dioxy (—OCH₂O—), sulfinyl (—SO—), sulfonyl (—SO₂—), or thio (—S—);

wherein any of the hydrocarbons can optionally be substituted with one or more amino, hydroxyl, cyano, nitro, (C₁-C₁₂)alkoxy, halo, trifluoro, trifluoro (C1-C₁₂)alkyl, NR¹R², or COOR¹; wherein R¹ and R² are each independently hydrogen, a (C₁-C₁₂) acyclic hydrocarbon or a (C₃-C₁₂) cyclic hydrocarbon.

The solvent 13 can be employed in any suitable amount, provided the amount of solvent 13 is effective to independently dissolve the adhesive 14 and the effective amount of solvent 13 remains stable in the formulation 5. In one embodiment, the stability is over a prolonged period of time, e.g., up to about 2 years, up to about 1 year, or up to about 6 months, typically experienced in the manufacturing, packaging, shipping, and/or storage of the patch 1.

In a specific embodiment, the nature and amount of the solvent 13 is selected, such that it will be generally recognized as safe (GRAS) for topical use.

### Adhesive

Any suitable adhesive 14 can be employed, provided the adhesive 14 provides the requisite adhesiveness to the patch 1 and the adhesive 14 remains stable in the formulation 5. In one embodiment, the stability is over a prolonged period of time, e.g., up to about 2 years, up to about 1 year, or up to about 6 months, typically experienced in the manufacturing, packaging, shipping, and/or storage of the patch 1. It is appreciated that the suitable adhesives 14 would be known to those skilled in the art. Suitable adhesives 14 are disclosed, e.g., in U.S. Pat. No. 4,675,009; U.S. Pat. No. 5,536,263; U.S. Pat. No. 4,696,854; U.S. Pat. No. 5,741,510, and references cited therein. In one embodiment the adhesive 14 is an acrylic ester copolymer.

Any suitable amount of adhesive 14 can be employed, provided the amount of adhesive 14 effectively provides the requisite adhesiveness to the patch 1 and the effective amount of the adhesive 14 remains stable in the formulation 5 over a prolonged period of time. Typically, the suitable amount of adhesive 14 depends upon the specific adhesive 14 or adhesives 14 employed. The formulation 5 can include an adhesive 14 in about 0.1 wt. % to about 50 wt. % of the formulation 5. In one embodiment, the formulation 5 can include an adhesive 14 in about 0.5 wt. % to about 10.0 wt. % of the formulation 5. In one embodiment, the formulation 5 can include an adhesive 14 in about 1.0 wt. % to about 15.0 wt. % of the formulation 5.

Alternatively, the adhesive 14 can include a hot melt pressure sensitive adhesive or solvent based pressure sensitive adhesive (e.g., polyacrylate, polyisobutylene, and polybutene), rubber, silicone based pressure sensitive adhesives (e.g., polydimethylsiloxane and resin mixtures), polystyrene-polybutadiene-polystyrene, polystyrene-polyisoprene-polystyrene, polystyrene-poly(ethylene-butylene)-polystyrene block polymers, or any combination thereof. In addition, the adhesive 14 can include a resin emulsion adhesive, wherein the resin emulsion adhesive can include vinyl acetate resin, acrylic ester copolymer, vinyl actetate/diocyl maleate copolymer, acrylic copolymer, or any combination thereof.

Other suitable adhesives 14 are disclosed, e.g., in U.S. Pat. Nos. 4,675,009; 5,536,263; 4,696,854; 5,741,510; and references cited therein.

The adhesive 14 can be located on and in any portion of the formulation 5. In one embodiment, the adhesive 14 can be located on the entire skin contact side of the formulation 5. When the adhesive skin patch 1 is placed upon the skin surface of a subject, the adhesive 14 in this configuration is in continuous contact with the skin surface of the subject.

In a specific embodiment, the nature and amount of the adhesive 14 is selected, such that it will be generally recognized as safe (GRAS) for topical use.

### Polymers

The formulation 5 can optionally include one or more polymers 9. The polymer 9 provides structure and strength to the adhesive 14 or can contain and release the active antimicrobial agent in a second formulation. Any suitable polymer 9 can be employed, provided the polymer 9 provides structure and strength to the adhesive 14 and the polymer 9 remains stable in the formulation 5. In one embodiment, the stability is over a prolonged period of time, e.g., up to about 2 years, up to about 1 year, or up to about 6 months, typically experienced in the manufacturing, packaging, shipping, and/or storage of the patch 1.

The suitable amount of polymer 9 can depend upon the specific polymer 9 or polymers 9 employed. For example, gum karaya can be employed as the polymer 9 in about 10 wt. % to about 55 wt. % of the formulation 5, in about 20 wt. % to about 35 wt. % of the formulation 5, or in about 23 wt. % to about 29 wt. % of the formulation 5. In one embodiment, gum karaya can be employed as the polymer 9 in about 24 wt. % to about 28 wt. % of the formulation 5.

Suitable polymers 9 include, e.g., starch, starch derivatives, polyvinyl pyrrolidone, polyethylene oxide, polyacrylate quats, polymaleic acid, polymaleic anhydride, polyurethanes, polyureas, gum karaya, gum acacia, locust bean gum, xanthan gum, guar gum, modified guar gum, maltodextrin, carboxymethyl cellulose, carboxypropyl cellulose, polyacrylamide, polyvinyl alcohol, poly AMPS, and polyacrylates. Other suitable polymers 9 are disclosed, e.g., in U.S. Pat. Nos. 4,675,009; 5,536,263; 4,696,854; 5,741,510; and references cited therein. In one embodiment, the polymer 9 is gum karaya. The term “gum karaya” refers to a vegetable gum produced as an exudate by trees of the genus Sterculia. Chemically, gum karaya is an acid polysaccharide composed of the sugars galactose, rhamnose, and galacturonic acid.

In a specific embodiment, the nature and amount of the polymer 9 is selected, such that it will be generally recognized as safe (GRAS) for topical use.

### Humectant

The formulation 5 can optionally include one or more humectants 17 to provide a moistening effect to the adhesive 14. For example, the humectant 17 can hydrate the polymer 9. Any suitable humectant 17 can be employed, provided the humectant 17 effectively provides a moistening effect to the adhesive 14 and the humectant 17 remains stable in the formulation 5. In one embodiment, the stability is over a prolonged period of time, e.g., up to about 2 years, up to about 1 year, or up to about 6 months, typically experienced in the manufacturing, packaging, shipping, and/or storage of the patch 1. One suitable humectant 17 is glycerin. Other suitable humectants 17 include polyhydric alcohols such as ethylene glycol, propylene glycol, triethylene glycol, tetraethylene glycol, and sorbitol.

Any suitable amount of humectant 17 can be employed, provided the amount of humectant 17 effectively provides a moistening effect to the adhesive 14 and the effective amount of humectant 17 remains stable in the formulation 5. The suitable amount of humectant 17 can depend upon the specific humectant 17 or humectants 17 employed and the specific polymer 9 or polymers 9 employed. For example, gum karaya can be employed as the polymer 9 and glycerin can be employed as the humectant 17 in about 20 wt. % to about 70 wt. % of the formulation 5, and in one embodiment about 30 wt. % to about 60 wt. % of the formulation 5, or about 40 wt. % to about 50 wt. % of the formulation 5.

In a specific embodiment, the nature and amount of the humectant 17 is selected, such that it will be generally recognized as safe (GRAS) for topical use.

### Topical Moisturizer

The formulation 5 can optionally include a topical moisturizer 18 (e.g., skin protectant). Any suitable topical skin protectant can be employed, provided the skin is effectively protected or moisturized and the skin protectant remains stable in the formulation 5. In one embodiment, the stability is over a prolonged period of time, e.g., up to about 2 years, up to about 1 year, or up to about 6 months, typically experienced in the manufacturing, packaging, shipping, and/or storage of the patch 1. Suitable skin protectants include, e.g. aloe, lanolin, glycerin, calamine, Vitamin E, Vitamin E acetate, Vitamin C, allantoin, aluminum hydroxide gel, bismuth subnitrate, boric acid, calamine, cocoa butter, dimethicone, glycerin, kaolin, live yeast cell derivative, petrolatum, pyridoxine hydrochloride, shark liver oil, sodium bicarbonate, sulfur, tannic acid, topical starch, trolamine, white petrolatum, zinc acetate, zinc carbonate zinc oxide, zinc sulfate, shea butter, and any combination thereof.

As used herein, calamine is a pink powder of zinc oxide and a skin protectant containing about 98% zinc oxide and about 0.5% ferric oxide; aloe is the dried latex of leaves of Curaco Aloe (Aloe barbadenis Miller, Aloe vera Linne) or Cape Aloe (Aloe ferox Miller and hybrids), of the family Liliacaea; Vitamin E is 3,4-dihydro-2,5,7,8-tetramethyl-2-(4,8,12-trimethyltridecyl)-2H-1-benzopyran-6-ol; Vitamin E acetate is 3,4-dihydro-2,5,7,8-tetramethyl-2-(4,8,12-trimethyltridecyl)-2H-1-benzopyran-6-ol acetate; and lanolin is the fat-like secretion of the sebaceous glands of sheep (i.e., complex mixture of esters and polyesters of 33 high molecular weight alcohols and 36 fatty acids) which is deposited onto the wool fibers. In one embodiment, the topical moisturizer 18 can be aloe and Vitamin E.

Aloe is commercially available as Aloe Vera Gel from Terry Laboratories (Melbourne, Fla.). Aloe Vera Gel is commercially available as Aloe Vera Gel 40× (20.0 wt. % solution in water), Aloe Vera Gel 1× (0.5 wt. % solution in water), Aloe Vera Gel 10× (5.0 wt. % solution in water), or solid Aloe Vera. The solid Aloe Vera can be dissolved in a carrier, such as water, to the desired concentration. In addition, the commercially available forms of Aloe Vera are optionally available as decolorized Aloe Vera.

Any suitable amount of topical moisturizer 18 can be employed, provided the suitable amount of topical moisturizer 18 or skin protectant effectively protects or moisturizes the skin and the effective amount of skin protectant remains stable in the formulation 5 over a prolonged period of time. The suitable and effective amount of topical moisturizer 18 can depend in part upon the specific moisturizer 18 or moisturizers 18 present in the formulation 5. For example, Aloe Vera Gel, 10× can be present up to about 40.0 wt. % of the formulation 5. In one embodiment, Aloe Vera Gel, 10× can be present up to about 5.0 wt. % of the formulation 5. In one embodiment, Aloe Vera Gel, 10× can be present up to about 1.0 wt. % of the formulation 5. In addition, Vitamin E acetate can be present up to about 5 wt. % of the formulation 5. In one embodiment, Vitamin E acetate can be present up to about 1.0 wt. % of the formulation 5. In one embodiment, Vitamin E acetate can be present up to about 0.5 wt. % of the formulation 5.

In a specific embodiment, the nature and amount of the topical moisturizer 18 is selected, such that it will be generally recognized as safe (GRAS) for topical use.

### Polyhydric Alcohol

The formulation 5 can optionally include one or more polyhydric alcohols 22. Suitable polyhydric alcohols 22 include, e.g., ethylene glycol, propylene glycol, triethylene glycol, tetraethylene glycol, sorbitol, or any combination thereof. Specifically, the polyhydric alcohol 22 can include propylene glycol.

Any suitable amount of polyhydric alcohol 22 can be employed. For example, when present in the formulation 5, the polyhydric alcohol 22 can be present up to about 35 wt. % of the formulation 5, up to about 15 wt. % of the formulation 5, or up to about 5 wt. % of the formulation 5. In one embodiment, the polyhydric alcohol 22 can be present in about 0.5 wt. % to about 5.0 wt. % of the formulation 5.

### Water

The formulation 5 can optionally include water, e.g., deionized water (DI). Any suitable amount of water can be employed, provided the amount of water maintains the adhesiveness of the adhesive 14 and maintains the appropriate stability of the formulation 5. For example, deionized water can be present up to about 50 wt. % of the formulation 5, up to about 40.0 wt. % of the formulation 5, or up to about 30.0 wt. % of the formulation 5. In one embodiment, deionized water can be present up to about 20.0 wt. % of the formulation 5. In one embodiment, deionized water can be present up to about 10.0 wt. % of the formulation 5. In one embodiment, deionized water can be present in about 5.0 wt. % to about 15.0 wt. % of the formulation 5.

### The Patch

The adhesive skin patch 1 can have any suitable size and shape. In addition, the adhesive skin patch 1 can be cut, as desired, to provide an adhesive skin patch 1 of a suitable size and shape. The adhesive skin patch 1 can be cut with any suitable cutting device such as a scissors, scalpel, or knife.

In one embodiment, the adhesive skin patch 1 has a length of about 0.1 inch to about 12 inches (about 2.54 mm to about 304.8 mm), about 0.1 inch to about 8 inches (about 2.54 mm to about 203.2 mm), of about 0.20 inch to about 4 inches (about 5.08 mm to about 101.6 mm), or about 0.2 inches to about 2.0 inches (about 5.08 mm to about 50.8 mm). In one embodiment, the adhesive skin patch 1 has a length of about 1.0 inch to about 8 inches (about 25.4 mm to about 203.2 mm), about 2 inches (about 50.8 mm to about 152.4 mm) to about 6 inches(about 5.08 mm to about 152.4 mm), or about 3 inches to about 4 inches (about 76.2 mm to about 101.6 mm).

In one embodiment, the adhesive skin patch 1 has a width of about 0.1 inch to about 12.0 inches (about 2.54 mm to about 304.8 mm), about 0.1 inch to about 4 inches (about 2.54 mm to about 101.6 mm), about 0.20 inches to about 2.0 inches (about 5.08 mm to about 50.8 mm), or about 0.2 inches to about 1.0 inch (about 5.08 mm to about 25.4 mm). In one embodiment, the adhesive skin patch 1 has a width of about 1.0 inch to about 8 inches (about 25.4 mm to about 203.2 mm), about 2 inches to about 6 inches (about 50.8 to about 152.4 mm), or about 3 inches to about 4 inches(about 76.2 mm to about 101.6 mm).

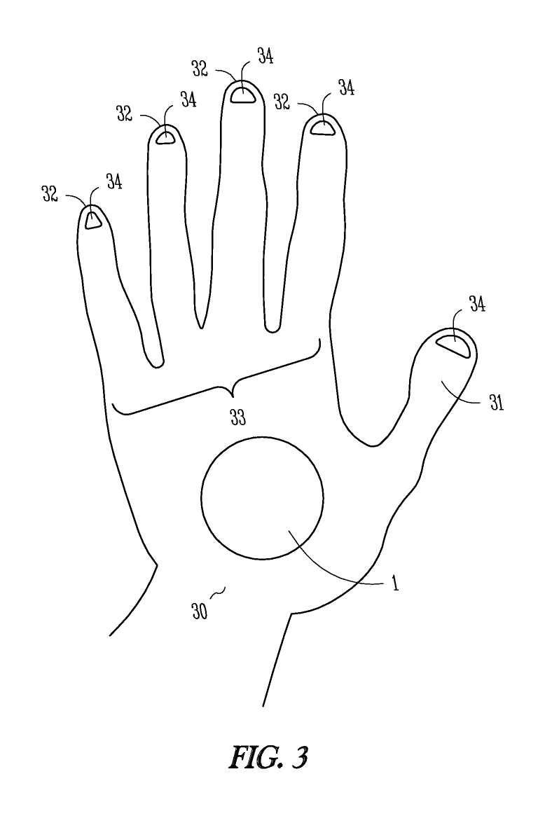

In one specific embodiment of the present invention, the adhesive skin patch 1 can be oval or elliptical in shape (see, FIG. 3). The oval or elliptical patch 1 can have a length of about 0.25 inches to about 0.50 inches (6.35 mm to about 12.7 mm) and a width of about 0.25 inches to about 0.50 inches (6.35 mm to about 12.7 mm). See, FIG. 3. In another specific embodiment of the present invention, the adhesive skin patch 1 can have a circular shape. The circular patch 1 can have a diameter of about 0.25 inches to about 0.50 inches (6.35 mm to about 12.7 mm).

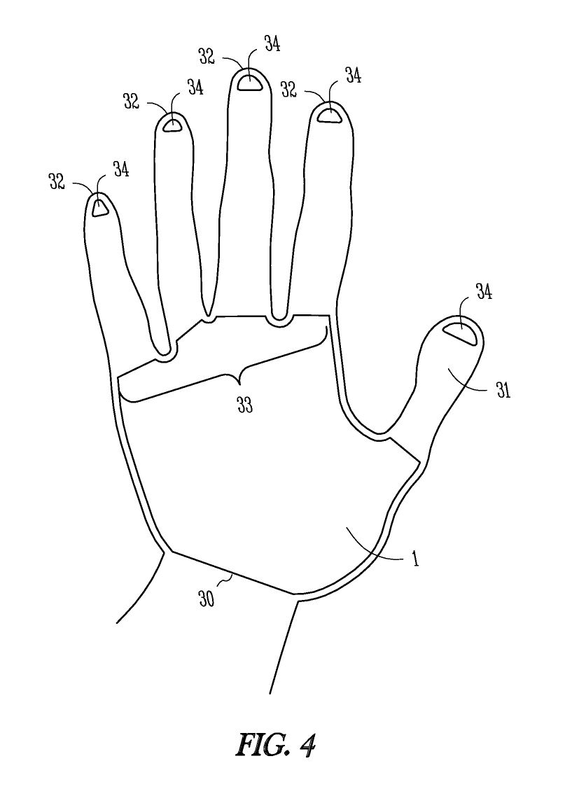

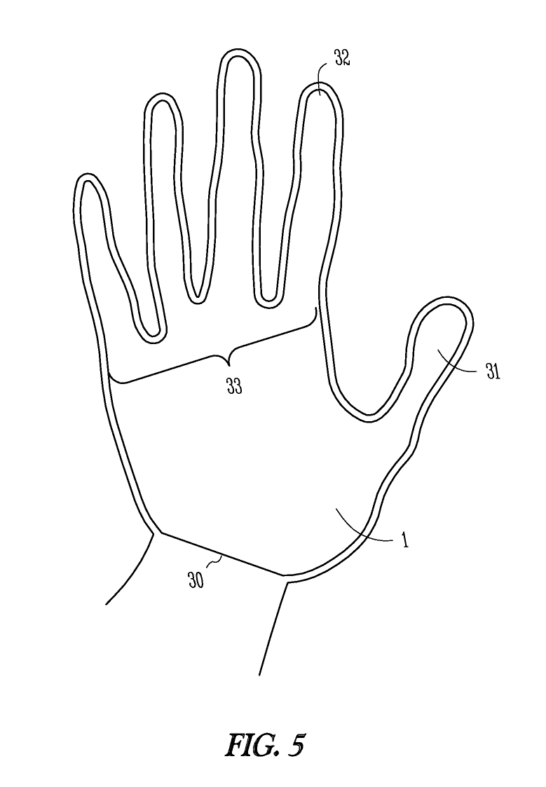

In another specific embodiment of the present invention, the adhesive skin patch 1 can be in the shape of the dorsal portion of a hand between the wrist 30 and the knuckles 33 (see, FIG. 4).

In another specific embodiment of the present invention, the adhesive skin patch 1 can be in the shape of the entire dorsal portion of a hand from the wrist 30 to the finger tips 32 and tip of the thumb 31, including the finger nails 34 (see FIG. 5)

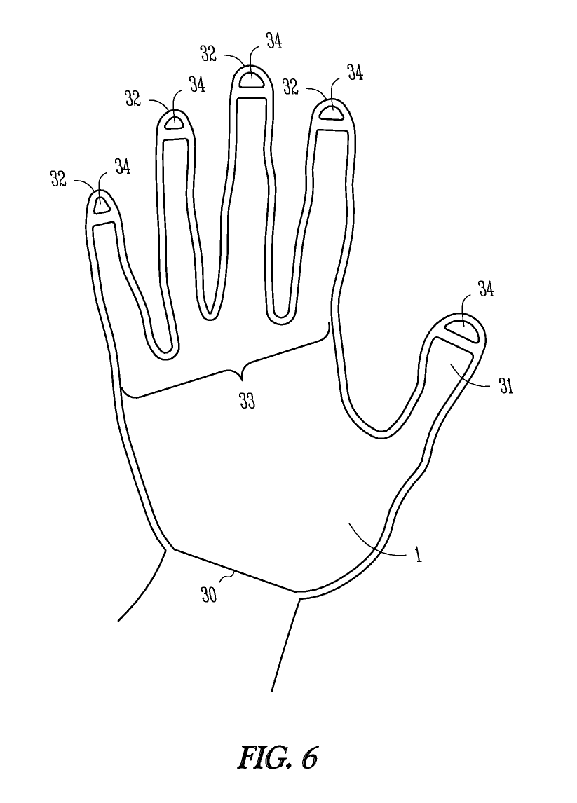

In another specific embodiment of the present invention, the adhesive skin patch 1 can be in the shape of the dorsal portion of a hand from the wrist 30 up to the finger nails 34, but not including the finger nails 34 (see FIG. 6). In one specific embodiment, the adhesive skin patch 1 can be manufactured in right handed and left handed versions, so as to fit on the dorsal portions of the right and left hand of the wearer.

In one embodiment, the adhesive skin patch 1 can be individually wrapped. Some consumers have shown a preference for adhesive skin patches that are individually wrapped. The individually wrapped adhesive skin patch 1 offers to the consumer the ability and convenience of being able to carry a few (e.g., 1, 2, or 3) adhesive skin patches 1 that are each individually wrapped. In such an embodiment, the use of one patch will not compromise the cleanliness and/or sterility of the remaining patches. Alternatively, more than one adhesive skin patch 1 can be wrapped together. For example, 2 to about 20, 2 to about 15, or 2 to about 10 adhesive skin patches 1 can be wrapped together. The cost of such packaging and wrapping can be decreased, compared to skin patches 1 that are individually wrapped. In one specific embodiment, the the adhesive skin patch can be individually wrapped as a right and left handed pair. The cost of having two or more patches wrapped together is typically less expensive than skin patches 1 that are individually wrapped.

In one embodiment of the present invention, the adhesive patch 1 is sterile. The adhesive patch 1 can be sterilized by any suitable means known to those of skill in the art. For example, the adhesive patch 1 of the present invention can be sterilized by irradiation. Specifically, the adhesive patch 1 of the present invention can be sterilized by terminal irradiation (e.g., when the adhesive patch 1 of the present invention is in the package).

### Production of the Patch

The adhesive patch 1 of the present invention can be formulated or manufactured employing the above components. The adhesive patch 1 of the present invention can be formulated or manufactured using any suitable technique. In one embodiment, the adhesive patch 1 can be formulated or manufactured as described in U.S. Pat. Nos. 5,536,263; 5,741,510; and references cited therein.

The adhesive patch 1 can be applied to any surface of a subject or to any surface of an article of clothing or a personal item worn by a subject. The subject can be a human (e.g., child that is younger than 18 years of age). The adhesive patch 1 can be applied by the subject, or by another person (e.g., parent). The subject can use the adhesive patch 1 by rubbing his/her hands across the back side 4 of the backing 2 of the patch 1. Rubbing the back side 4 of the backing 2 of the patch 1 can cause the antimicrobial 15 to come into contact with the hand that is rubbing. The antimicrobial 15 can then come into contact with the non-rubbing hand or another skin surface through subsequent rubbing. In one embodiment, the subject can wear two adhesive patches 1, one on the dorsal aspect of each hand.

The following examples are provided to illustrate the practice of the present invention and the invention is not meant to be limited thereby.

### EXAMPLES

### Example 1

### Antibacterial Efficacy Testing Using Polyhexamethylenebiguanide Antimicrobial

Excilon™ AMD™ Antimicrobial I.V. Sponges (by Kendall, part of Covidien, Inc). were purchased, and used for the subsequently described antibacterial efficacy testing. These sponges are composed of 0.2% polyhexamethylenebiguanide as active antimicrobial. The antimicrobial is bonded on non-woven cloth, the size of each cloth is 2 inch×2 inch (50.8 mm×50.8 mm).

### Protocol

The following protocol describes how antibacterial efficacy testing was conducted. This protocol also applies to Examples 2 and 3 below.

Assessment of Antibacterial Activity by Direct Contact “Kill of Treated Textiles Against Staphylococcus aureus Misting study.”

### Objective

This test was designed to evaluate the bactericidal effectiveness of treated textile materials in direct contact with Staphylococcus aureus. It determines the potential of textile material(s) impregnated with antimicrobial agents to inactivate bacteria on direct contact. The test is based on AATCC Test Method 100-2004.

### Testing Conditions

Several types of treated textile material, as well as non-treated control textile, were evaluated in this study. In addition, a liquid control without textile was performed using the same level of bacterial challenge to serve as another baseline control to measure the input bacterial loads. The test textile materials were challenged with Staphylococcus aureus by a misting procedure and held for a specified exposure (contact) time. Two contact time points were tested for each test textile, non-treated control textile, and liquid (no textile) control sample. Upon completion of the exposure period, surviving bacteria were extracted using an appropriate neutralizing recovery medium, serially diluted and cultured. One replicate run was performed for each condition.

### Materials

A. At our request, test, control, and reference substances were tested by MICROBIOTEST Laboratories, a division of MicroBac, (Sterling, Va.).
B. Materials supplied by MICROBIOTEST, included, but were not limited to:

1. Challenge microorganisms:
Staphylococcus aureus, ATCC 6538

2. Media and reagents:
a. Nutrient Broth (NB)
b. Nutrient Agar (NA)
c. DE Neutralizing Broth (DE)
d. Butterfield's Phosphate Buffered Dilution Water (PBDW)

3. Laboratory equipment and supplies including:
a. Sterile test tubes and bottles
b. Sterile flasks
c. Stomaching device and sterile bags

### Experimental Design

A. Inocula preparation:
   Bacteria from stock cultures were transferred into the nutrient broth (NB) and incubated. Daily transfers were made for at least one passage (but no more than for 10 days). Tubes of 10 mL NB were inoculated with one loopful of inoculum per tube and incubated. After 48-54 hours, the cultures were used for contaminating the carriers. If necessary, the culture was diluted to yield approximately 10⁵ to 10⁷ colony-forming units (CFU) per carrier for the initial counts control.
B. Test and control textile preparation:
   The test and non-treated control textiles were cut to approximately 2.5 inch×2.5 inch (if not pre-cut) (63.5×63.5 mm). All prepared materials, now designated as carriers were exposed to UV radiation, under the hood, for a minimum of 30 minutes per side prior to use. No other pre-treatment or pre-conditioning was performed.

For each inoculation, a sterile barrier, containing a 2 inch×2 inch opening (50.8 mm×50.8 mm), was placed over the carriers to ensure that the inoculum was only applied to the testing surface (and not to the sides, or underside of the carriers).
C. Test:
   Several types of treated textile materials were evaluated using two contact times for each. A single replicate run was evaluated for each condition.
   For each run, the challenge bacteria were added in the following manner: the inoculum were misted on using a spray device (Nalgene Aerosol Spray Bottle) from a distance of 4 inch-5 inch (101.6×127 mm) for two pumps, one second per pump. The volume of inoculum applied was between 0.3 and 0.4 mL.
   Once inoculated, each inoculated carrier was maintained at ambient room temperature for the designated contact time(s). At the conclusion of the contact period, each carrier was placed into a sterile stomacher bag containing 40 mL of neutralizing broth (DE) (equivalent to 10⁻¹ with a 4× multiplication factor) and was designated as the extraction sample. Each sample was processed in a stomacher for five minutes. A one mL aliquot of the extraction sample was serially diluted ten-fold in phosphate buffered dilution water (PBDW) as required. Duplicate aliquots from selected dilutions were pour plated using the nutrient agar (NA).
D. Incubation and enumeration:
   Upon completion of the test, all plates were inverted and incubated over two nights at 37° C.±2° C. Following incubation, all plates were removed, the colonies were counted and the colony forming units (CFU) recovered per carrier was determined.
E. Controls:

1 Non-treated Textile Control:
Non-treated textile carriers were evaluated as controls using the same procedures as the test carriers (one carrier was processed for each contact time).

2 Inoculum confirmation counts:
A one mL aliquot of prepared inoculum was serially diluted ten-fold in PBDW. Duplicate aliquots from selected dilutions were pour plated using NA. Plates were inverted and treated in the same manner as the test plates.

3. Application evaluation:
The prepared inoculum were misted into a tube using the same techniques employed for the test. The procedure was repeated two additional times with the misted inoculums delivered into the same initial tube. The inoculum was allowed to settle and the volume in the tube was measured and the average of the three repetitions was reported.

4. Liquid control:
One replicate, using a glass Petri dish, was tested for the liquid (i.e., no textile) control as the first baseline control. A sterile glass Petri dish was misted with bacteria in the same manner as the test carriers and a timer was started. After each contact time, 40 mL of DE was added to the Petri dish and mixed thoroughly by pipetting. The recovery solution was then be transferred to a stomacher bag and processed in the stomacher bag for five minutes as described above. An aliquot of the extraction sample was serially diluted ten-fold in phosphate buffered dilution water (PBDW) as required. Duplicate aliquots from selected dilutions were pour plated using NA.

5. Neutralizer effectiveness: (performed for each of three test material)
This control was included to demonstrate the absence of residual activity of the antimicrobial in the extraction tube. Duplicate textile carriers (noncontaminated) were placed in individual bags containing 40 mL of DE (extraction sample, equivalent to the 10⁻¹). One test textile carrier was gently mixed for 10 seconds; the second carrier was gently mixed for 5 minutes. Immediately after mixing, the carriers were removed and placed into a secondary bag containing 40 mL of DE. Fewer than 500 CFU of the prepared inoculum were added to each of the bags (extraction sample bag and the bag containing the subcultured carrier for each mixing time).
Within ten minutes, three mL from each bag were sampled and one mL aliquots were plated using NA. The total CFU added to each bag was confirmed using duplicate NA plates. The plates were treated in the same manner as the test plates.

6. Sterility control:
Duplicate plates of NA were incubated with the test. In addition, duplicate one-mL aliquots of PBDW and DE were plated using NA. These plates were incubated with the test.

7. Challenge microorganism confirmation:
In order to confirm growth consistent with the challenge microorganism, Gram stains were performed, from a representative colony from a nontreated textile control plate. If applicable, an isolated colony from a test plate was treated in the same manner and compared to the non-treated textile control stain. The colony morphology was noted as well.

### Test Acceptance Criteria:

The test was acceptable for evaluation of the test results if the criteria listed below were satisfied. No statistical methods were used in this test.

The neutralizer effectiveness controls must show comparable recovery (CFU/bag) to the confirmation counts.

### Data Presentation:

The results are presented as:
The log₁₀ reduction and percent reduction from the non-treated textile control; and
The results for all controls.

### Personnel and Testing Facilities:

At our request, this study was conducted at MICROBIOTEST (Sterling, Va.).

### Results:

Results are presented in Tables 1-3. The challenge microorganism was confirmed by colony morphology and Gram stain to be consistent with Staphylococcus aureus. The sterility control exhibited no growth. The viability and neutralizer effectiveness controls exhibited growth. The inoculum confirmation count control averaged 2.4×10⁷ colony forming units (CFU)/mL. The application evaluation control results averaged 0.37 mL. Percent reduction and Log₁₀ reduction (presented in Table 1) were calculated using the following equations:

$$\frac{Average Non \text{-} treated Control Sample - Treated Sample}{Average Non \text{-} treated Control Sample} \times 100 = Percent Reduction$$

$${Log}_{10} ( Non \text{-} treated control sample ) - {Log}_{10} ( Treated sample ) = {Log}_{10} ( Reduction )$$

TABLE 1
Test Results
Results Expressed as CFU per Carrier and Percent Reduction and
Log Reduction

| | Contact | | Percent | Log |
|---|---|---|---|---|
| Test Agent | Time | CFU/carrier | Reduction | Reduction |
| Kendall Excilon ™ | 3 min | 1.6 × 10⁴ | 98.93 | 1.97 |
| AMD Antimicrobial IV | 15 min | 2.0 × 10⁴ | 98.95 | 1.98 |
| Sponges (6 Ply 2 × 2) | | | | |

TABLE 2
Control Results
Results Expressed as CFU/carrier

| Test Agent | Contact Time | CFU/carrier |
|---|---|---|
| Untreated Polyester | 3 min | 1.5 × 10⁶ |
| | 15 min | 1.9 × 10⁶ |

TABLE 3
Liquid Control
Results Expressed as CFU/carrier

| Contact Time | CFU/carrier |
|---|---|
| 3 min | >3.0 × 10⁵ |
| 15 min | >3.0 × 10⁵ |

TABLE 4
Neutralizer Effectiveness Control
Results Expressed as Average Colony Forming Unit (CFU) per Bag

| Test Agent | Contact Time/Bag | Average Count |
|---|---|---|
| Kendall Excilon ™ | Extraction Bag (10 seconds) | 5.3 × 10¹ |
| AMD Antimicrobial IV | Carrier Bag (10 seconds) | 2.0 × 10¹ |
| Sponges (6 Ply 2 × 2) | Extraction Bag (5 minutes) | 2.7 × 10¹ |
| | Carrier Bag (5 minutes) | 2.0 × 10¹ |

### Example 2

### Antibacterial Efficacy Testing Using Quaternary Ammonium Silicone Antimicrobial (SiShield, Inc.) Treated 100% Polyester Textile (Testfabrics, Inc)

Polyester fabric samples (2 inch×2 inch; 50.8 mm×50.8 mm) were treated (Testfabrics Inc, 100% polyester fabric, style #749, lot #6284) by applying SiShield antimicrobial process. This material is based on quaternary ammonium silicone (“SiQac”) compounds with the following structural formula (obtained from http://www.sishield.com/chemistry.html).

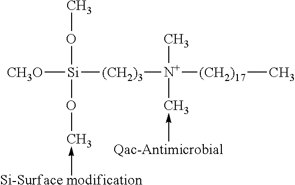

### Results:

Results are presented in Tables 5-8. The challenge microorganism was confirmed by colony morphology and Gram stain to be consistent with Staphylococcus aureus. The sterility control exhibited no growth. The viability and neutralizer effectiveness controls exhibited growth. The inoculum confirmation count control averaged 1.8×10⁷ colony forming units (CFU)/mL. The application evaluation control results averaged 0.37 mL. Percent reduction and Log₁₀ reduction (presented in Table 1) were calculated using the following equations:

$$\frac{Average Non \text{-} treated Control Sample - Treated Sample}{Average Non \text{-} treated Control Sample} \times 100 = Percent Reduction$$

$${Log}_{10} ( Non \text{-} treated control sample ) - {Log}_{10} ( treated sample ) = {Log}_{10} reduction$$

TABLE 5a
Test Results
Results Expressed as CFU per Carrier and Percent
Reduction and Log Reduction

| | Contact | | Percent | Log |
|---|---|---|---|---|
| Test Agent | Time | CFU/carrier | Reduction | Reduction |
| Quaternary | 1 min | 1.8 × 10⁴ | 99.56 | 2.36 |
| Ammonium | 3 min | 4.8 × 10³ | 99.73 | 2.57 |
| Silicone (SiShield) | | | | |
| treated 100% | | | | |
| Polyester Textile | | | | |
| (Testfabrics, Inc) | | | | |

A separate experiment was conducted using the same SiShield technology, however, using different contact times.

TABLE 5b

| | Contact | | Percent | Log |
|---|---|---|---|---|
| Test Agent | Time | CFU/carrier | Reduction | Reduction |
| Quaternary | 3 min | 1.2 × 10³ | 99.92 | 3.10 |
| Ammonium | 15 min | 3.8 × 10² | 99.98 | 3.70 |
| Silicone (SiShield) | | | | |
| treated 100% | | | | |
| Polyester Textile | | | | |
| (Testfabrics, Inc) | | | | |

TABLE 6
Control Results
Results Expressed as CFU per carrier

| Test Agent | Contact Time | CFU/carrier |
|---|---|---|
| Control (100% polyester, untreated) | 1 min | 4.1 × 10⁶ |
| | 3 min | 1.8 × 10⁶ |

TABLE 7
Liquid Control
Results Expressed as CFU per mL

| Contact Time | CFU/carrier |
|---|---|
| 1 min | 1.2 × 10⁶ |
| 3 min | 5.5 × 10⁵ |

TABLE 8
Neutralizer Effectiveness Control
Results Expressed as Average Colony Forming Unit (CFU) per bag

| Test Agent | Contact Time/Bag | Average Count |
|---|---|---|
| Quaternary | Extraction Bag (10 seconds) | 5.9 × 10² |
| Ammonium | Carrier Bag (10 seconds) | 4.5 × 10² |
| Silicone (SiShield) | Extraction Bag (5 minutes) | 5.1 × 10² |
| treated 100% | Carrier Bag (5 minutes) | 4.3 × 10² |
| Polyester Textile | Carrier Bag (10 seconds) | 3.9 × 10² |
| (Testfabrics, Inc) | Extraction Bag (5 minutes) | 4.9 × 10² |
| | Carrier Bag (5 minutes) | 4.9 × 10² |

Neutralizer Effectiveness Average Confirmation Counts: 4.9 × 10² CFU
SiShield technology applied to a polyester fabric (100%, Testfabrics, Inc) resulted in 2-3 times reduction of bacterial counts after only a few minutes (e.g., 1 min, 3 min, 15 min).

### Example 3

### Antibacterial Efficacy Testing Aegis Antimicrobial Treated 100% Polyester Textile (Testfabrics, Inc)

Polyester fabric samples (2 inch×2inch; 50.8×50.8 mm) were treated (Testfabrics Inc, 100% polyester fabric, style #749, lot #6284) by Aegis Microbeshield with 0.2% active AEM (from AEM5772) or with with 0.2% active AEM (from AEM5772)+2% AMMS-1. AMMS-1 is a durable wicking agent that is compatible with the AEGIS molecule.

### Results:

Results are presented in Tables 9-13. The challenge microorganism was confirmed by colony morphology and Gram stain to be consistent with Staphylococcus aureus. The sterility control exhibited no growth. The viability and neutralizer effectiveness controls exhibited growth. The inoculum confirmation count control averaged 1.8×10⁷ colony forming units (CFU)/mL. The application evaluation control results averaged 0.37 mL. Percent reduction and Log₁₀ reduction (presented in Table 1) were calculated using the following equations:

$$\frac{Average Non \text{-} treated Control Sample - Treated Sample}{Average Non \text{-} treated Control Sample} \times 100 = Percent Reduction$$

$${Log}_{10} ( non \text{-} treated control sample ) - {Log}_{10} ( treated sample ) = {Log}_{10} ( reduction )$$

TABLE 9
Test Results
Results Expressed as CFU per Carrier and Percent
Reduction and Log Reduction

| | Contact | | Percent | Log |
|---|---|---|---|---|
| Test Agent | Time | CFU/carrier | Reduction | Reduction |
| 0.2% AEM5772 plus | 1 min | 5.6 × 10⁵ | 60.00 | 0.40 |
| and 0.2% AMMS | 3 min | 4.3 × 10³ | 98.95 | 1.98 |
| (Aegis, Inc.) | | | | |

TABLE 10
Control Results
Results Expressed as CFU per carrier

| Test Agent | Contact Time | CFU/carrier |
|---|---|---|
| Control Sample 1 (treated only | 1 min | 1.4 × 10⁶ |
| with water and cured) | 3 min | 4.1 × 10⁵ |

TABLE 11
Liquid Control
Results Expressed as CFU per mL

| Contact Time | CFU/carrier |
|---|---|
| 1 min | 1.2 × 10⁶ |
| 3 min | 5.5 × 10⁵ |

TABLE 12
Neutralizer Effectiveness Control
Results Expressed as Average Colony Forming Unit (CFU) per bag

| Test Agent | Contact Time/Bag | Average Count |
|---|---|---|
| 0.2% AEM5772 plus | Extraction Bag (10 seconds) | 6.1 × 10² |
| and 0.2% AMMS | Carrier Bag (10 seconds) | 3.9 × 10² |
| (Aegis, Inc.) | Extraction Bag (5 minutes) | 4.9 × 10² |
| | Carrier Bag (5 minutes) | 4.9 × 10² |

Neutralizer Effectiveness Average Confirmation Counts: 4.9×10² CFU

The Aegis' technology treated polyester samples were associated with an up to 2× log reduction of bacterial counts.

### Example 4

### Virucidical Effectiveness of Quaternary Ammonium Silicone (SiShield) Treated 100% Polyester Textile (Testfabrics, Inc.)

### Protocol

Assessment of Virucidal Effectiveness via Direct Contact Kill by Treated Textile—Human Influenza A Virus

### Misting Study

### Objective

This test was designed to evaluate the virucidal effectiveness of treated textile material against Human Influenza A Virus. The test determines the potential of the textile material impregnated with antimicrobial agents to inactivate the virus via direct contact. The test was designed to simulate consumer use and is based on the AATCC Test Method 100-2004 with customization for virus testing.

### Testing Conditions

One type of treated textile and one type of non-treated control textile, one lot each, was tested in this study. The textiles were challenged with virus by a misting procedure and held for a specified exposure (contact) time. Two contact time points were tested for each test and control textile. Upon completion of the exposure period, the surviving virus were extracted using an appropriate recovery medium, serially diluted and inoculated onto the host cell plates to determine the amount of infectious virus. The recovered viral load from the treated textile were compared with that from the non-treated control textile to determine the viral reduction efficacy of treated (test) textile. One replicate run was performed for each condition. No liquid (i.e., no textile) control was performed for this study.

### Materials

A. Test, control, and reference substances used a 100% polyester fabric (Testfabrics, Inc., Filament Polyester Oxford Weave).
   Samples were treated by applying SiShield antimicrobial process. This material is based on quaternary ammonium silicone (“SiQac”) compounds. The structure of these compounds is shown above in Example 2.
B. Materials supplied by MICROBIOTEST, including, but not limited to:

1. Challenge virus Human Influenza A Virus, A/PR/8/34 (H1N1)

2. Host: MDCK cells

3. Mister: Thermo Scientific, Nalgene Aerosol Spray Bottle

4. Other laboratory equipment and supplies

5. Media and reagents

### Experimental Design

All of the procedures involved in performance of this study are described in a detailed series of SOPs that are maintained at MICROBIOTEST SOPs and Logs are referred to in the raw data and are required as part of GLP regulations.

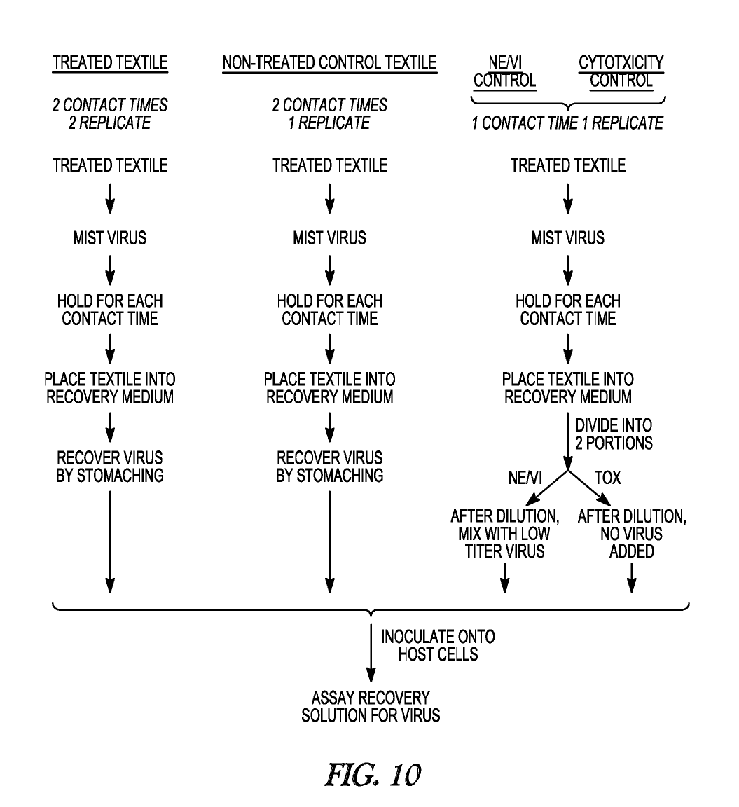

The study flow diagram is summarized in FIG. 10, with details described below.
A. Inoculum preparation:
   Viral stocks were purchased from reputable sources that identify them by scientifically accepted methods and may have been propagated at MICROBIOTEST. Records were maintained that demonstrate the origin of the virus. The virus stocks were stored at an ultra-low temperature.
   Frozen viral stocks were thawed on the day of the test (fresh stock cultures may also be used. High-titered virus stock (at least 106.5 TCID50/mL) was used to ensure a minimum of 3 to 4 log-reduction window for the treated textile. If the original ‘vial of virus was to be diluted, an appropriate dilution medium such as 0.1× MEM, was used to retain virus infectivity without introducing excessive extra organic load.
B. Test material preparation and pre-conditioning:
   Each test or control textile was cut to approximately 2.5 inch×2.5 inch (63.5 mm×63.5 mm) size (if not pre-cut). All carriers were exposed to UV light, under a hood, for a minimum of 30 minutes per side prior to use to reduce the bioburden. No other pre-treatment or pre-conditioning was performed.
   For each viral inoculum application, a sterile barrier, containing approximately a 2 inch×2 inch (50.8 mm×50.8 mm) opening was placed over the piece of test textile material to ensure that the inoculum was applied only to the testing surface and not to the sides or the underside of the carriers.
C. Test
   One type of treated textile material and one type of non-treated control textile were evaluated each at two contact times and one replicate. No liquid control was performed in this study.
   For each run, the challenge virus was added in the following manner: The inoculum was misted on using a spray device (Nalgene Aerosol Spray Bottle) from a distance of 4 inches to 5 inches (101.6 mm×127.0 mm) for two pumps, one second per pump. The volume of inoculum applied should be between 0.3 and 0.4 mL and not exceeding 0.4 mL. The average amount of inoculum applied from three medium challenge runs was measured and recorded.
   After misting, the Textile piece was held for the specified contact time. Upon completion of the contact time, the carrier was immediately placed into a sterile stomacher bag containing 40 mL of neutralizer (extraction medium). Each sample was processed in the stomacher bag for five minutes to extract virus. An aliquot of the extraction sample was immediately collected. The sample was then ten-fold serially diluted in dilution medium and inoculated onto host cells.
   When Sephacryl columns (a size-exclusion chromatography column) were used to further reduce the cytotoxicity of the extracted sample, the extraction samples were all processed by the column (except the Column Titer Control, see Section E.5, below). Specifically, an aliquot of each sample was loaded into separate pre-spun Sephacryl S-1000 columns and spun at approximately 1000 rpm for 4 minutes. The eluates were aseptically collected and serially ten-fold diluted and processed as described below.
D. Infectivity assay:
   The residual infectious virus in the test and controls was detected by viral-induced cytopathic effect (CPE).
   Selected dilutions of the neutralized inoculum/test agent mixture were added to cultured host cells (at least four wells per dilution, per reaction mixture) and incubated at 33° C.±2° C. with 5±1% CO₂ for a period of 7-9 days. The host cells were washed twice with phosphate buffered saline (PBS) prior to sample inoculation. The host cell cultures were observed and refed, as necessary, during the incubation period. These activities, if applicable, were recorded. Then the host cells were examined for presence of infectious virus. The resulting virus-specific cytopathic effects and test agent-specific cytotoxic effects were scored by examining both test and controls. These observations were recorded.
E. Controls:

1. Non-treated control textile:
As described in Section C, Test above, one replicate, using an untreated control textile containing no active ingredient(s), was tested along with the treated textiles at each of the two contact times. Each carrier was inoculated with virus in the exact same manner as the test carriers. After the designated contact time, the carrier was placed into a sterile stomacher bag containing 40 mL of neutralizer (extraction medium). The sample was processed in the stomacher bag for five minutes. After the stomaching process, the carrier was immediately removed, and an aliquot of the extraction sample was serially ten-fold diluted in dilution media.
This control determines relative loss of virus infectivity from exposure to the non-treated textile, neutralization, and stomaching process alone. The viral load from this control is used as the baseline to determine the viral reduction by the treated textile via direct contact kill.
2. Neutralizer effectiveness/viral interference (NE/VI) control:
   This control is included to determine if residual active ingredient is present after neutralization and if the neutralized test agent interferes with virus infectivity. One replicate was performed for this control.
   Treated textile was misted with medium in lieu of virus and held for a contact period. After the contact time (the longer contact time was used as a worst case scenario), the carrier was placed in a sterile stomacher bag containing 40 mL neutralizer (extraction medium) and stomached for five minutes. Then the carrier was removed and discarded.
   Two aliquots of the extraction sample, one for the cytotoxicity control and one for the neutralizer effectiveness/viral interference control, were transferred to tubes containing dilution medium and serially ten-fold diluted.
   For the Neutralizer effectiveness/viral interference control, following serial dilutions, 100 μL of a low titered virus was added to 4.5 mL of each selected dilution (typically Undiluted, 10⁻¹ and 10⁻² of the extraction sample) and held for a period greater than or equal to the contact time. These samples were then used to inoculate host cells as described for the test procedure and incubated in the same manner as the test.

3. Cytotoxicity (TOX) control:
This control evaluated the cytotoxicity of the neutralized extraction sample to the host cells. One replicate was performed for this control.
Selected dilutions (typically Undiluted, 10⁻¹ and 10⁻² of the extraction sample) obtained from the Neutralizer effectiveness/viral interference control run were inoculated onto host cells and incubated together with other test and control samples as described for the test procedure. The condition of the host cells was recorded at the end of the incubation period. These effects must be distinct from virus-specific cytopathic effects (CPE).

4. Volume application evaluation:
The virus resuspension medium (0.1× MEM) was misted into a dish using the same techniques employed for the test at three repetitions. The medium was misted into three independent dishes. The misted medium was allowed to settle and the volume in each dish was measured and reported. The average volume of challenge per run was one third of the total volume from these three runs.

5. Cell viability control:
At least four cells were inoculated with an appropriate medium during the incubation phase of the study.
This control demonstrates that the cells remain viable throughout the course of the assay period. In addition, it confirms the sterility of the media employed throughout the assay period.

6. Virus Stock Titer control (VST)
An aliquot of the virus inoculum used in the study was directly serially diluted and inoculated onto the host cells to confirm the titer of the stock virus. This control demonstrates that the titer of the stock virus is appropriate for use and that the viral infectivity assay was performed appropriately.
F. Calculation:
   The 50% tissue culture infectious dose per mL (TCID50/mL) was determined using the method of Spearman-Karber (Spearman C and Kaerber G In: Bibrack B, Wittmann G, eds. Virologische Arbeitsmethoden, Stuttgart: Fischer Verlag, 1974, pp 37-39).
   The test results are reported as the reduction of the virus titer due to treatment with test agent expressed as log₁₀. Viral reduction was calculated from the non-treated control textile baseline.

### Testing Facility

At our request, this study was conducted at MICROBIOTEST (Sterling, Va.).

### Results

Antiviral efficacy testing using polyester textile treated with quaternary ammonium silicone (SiShield) are presented in Tables 13-15.

The Viral load was determined in the following manner:

Viral Load (Log₁₀TCID50)=Titer (Log₁₀TCID50/mL)+Log₁₀[Volume (mL)]

The log₁₀ Reduction Factor (LRF) was calculated in the following manner:

Log₁₀ Reduction Factor=Initial viral load (Log₁₀TCID50)−Output viral load (Log₁₀TCID50)

The 95% Confidence Interval (CI) for the LRF are calculated as follows:

(CI LRF)²=(CI input)²+(CI output)²

When all negatives were observed, simply replace the output load by c×Output Volume for calculating the log reduction, where c was taken from the Poisson 95% confidence interval discussed above, and substitute 0 for CI output in calculating the 95% confidence interval of the log reduction factor.

TABLE 13
Titer Results

| | | Titer ± 95% | | Viral Load |
|---|---|---|---|---|
| | Contact | CI (Log₁₀ | Volume | (Log₁₀ |
| Sample | Time | TCID50/ml) | (mL) | TCID 50) |
| Virus Stock Titer | NA | 6.75 ± 0.25 | NA | NA |
| Control | | | | |
| Cell viability/media | NA | No virus was detected, cells were viable; | | |
| sterility control | | media were sterile | | |
| Volume application | NA | Average volume of challenge: 0.39 mL | | |
| evalutation | | per replicate | | |
| Polyester textile | 1 min | 6 ± 0.28 | 40 | 7.6 ± 0.28 |
| control (untreated) | | | | |
| Polyester textile | 3 min | 6.25 ± 0.25 | 40 | 7.85 ± 0.25 |
| control (untreated) | | | | |
| Quaternary | 1 min | 3.25 ± 0.47 | 40 | 4.85 ± 0.47 |
| Ammonium | | | | |
| Silicone (SiShield) | | | | |
| treated 100% | | | | |
| Polyester Textile | | | | |
| (Testfabrics, Inc.) | | | | |
| Quaternary | 3 min | 2.5 ± 0.0 | 40 | 4.1 ± 0.0 |
| Ammonium | | | | |
| Silicone (SiShield) | | | | |
| treated 100% | | | | |
| Polyester Textile | | | | |
| (Testfabrics, Inc.) | | | | |

TABLE 14
Neutralizer Effectives/Viral Interference and Cytotoxicity Controls
Quaternary Ammonium Silicone (SiShield) treated 100% Polyester Textile
(Testfabrics, Inc.) (Contact time = 3 minutes)

| Dilution of the | Neutralizer | |
|---|---|---|
| Neutralized | Effectiveness/Viral | Cytotoxicity |
| Sample | Interference Control | Control |
| Undiluted | Cytotoxicity observed/Viral | Cytotoxicity |
| | Cytopathic effect could not be | observed |
| | evaluated | |
| 10⁻¹ | Virus detected in 4 out of | No cytotoxicity |
| | 4 wells | observed |
| 10⁻² | Virus detected in 4 out of | No cytotoxicity |
| | 4 wells | observed |

TABLE 15
Viral Reduction (based on 100% untreated polyester control textile)

| | | Initital Viral | Output Viral | |
|---|---|---|---|---|
| | Contact | Load(Log₁₀ | Load (Log₁₀ | Log₁₀ |
| Sample | Time | TCID50) | TCID50) | Reduction |
| Quaternary | 1 min | 7.6 ± 0.28 | 4.85 ± 0.47 | 2.75 ± 0.55 |
| Ammonium | | | | |
| Silicone (SiShield) | | | | |
| treated 100% | | | | |
| Polyester Textile | | | | |
| (Testfabrics, Inc.) | | | | |
| Quaternary | 3 min | 7.85 ± 0.25 | 4.1 ± 0.0 | 3.75 ± 0.25 |
| Ammonium | | | | |
| Silicone (SiShield) | | | | |
| treated 100% | | | | |
| Polyester Textile | | | | |
| (Testfabrics, Inc.) | | | | |

A very significant 2.75 to 3.75 log₁₀ reduction was observed at 1 min and 3 min contact time respectively.

### Example 5

### Construction of a Hand Sanitizer Patch

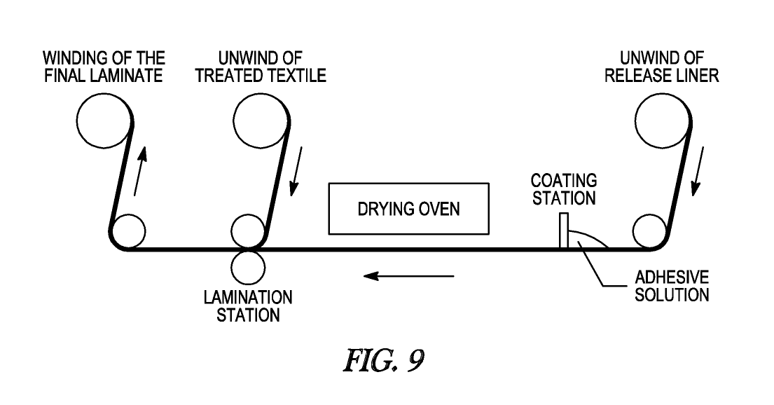

A schematic of the manufacturing process is shown in FIG. 9.

A roll of polyester fabric (100%, Filament Polyester Oxford Weave, by Testfabrics, Inc., style #749, lot #6284) was treated by applying SiShield antimicrobial process. This material is based on quaternary ammonium silicone (“SiQac”) compounds. The structure of these compounds is shown above in Example 2.

The process of manufacturing lamination of the polyester fabric and the release liner were done on the coating machine. The coating machine consists of the coating head station where the liquid adhesive solution (DuroTak 87-900A adhesive, an acrylic type non-functional with 42$ solids, Henkel AG) was residing, a hot air oven and the lamination station. The web of release liner (3 mil; 75 micron polyethylene terephthalate (PET) siliconized on one side) when moving in the oven direction was used to spread the adhesive on the release liner at the thickness determined by the gap of the coating knife. The adhesive was exposed to drying air impinging on the adhesive surface in the oven causing evaporation of solvents leaving semisolid adhesive polymer layer. After exiting the oven the release liner was overlaid with the textile web and laminated together between two lamination rollers. The laminate consisted of the textile material, the adhesive layer attached now to the polyester and to the release liner. The laminate was wound and later cut to 2 inch×2 inch (50.8 mm×50.8 mm) patches on a separate die cutting machine.

It is understood that other test methods can be used to evaluate the efficacy of this invention. In principal, there are two kinds of test methods:
1) Test methods focusing on products to remove transient flora
2) Test methods focusing on products to remove resident flora

The majority of test methods of products for removing transient flora involve artificial contamination of the volunteer's skin with a defined amount of a test organism before the volunteer uses the hand sanitizer product. Surgical hand scrubs are however tested for their ability to remove resident flora without contaminating volunteers' hands.
1. Misting study: for evaluation and selection of dry technologies (AATCC-100 standard). This method was used in the Examples above.
2. Wipe study: The towelette method is based on AOAC Germicidal Spray Products as Disinfectants standard (Official Methods of Analysis, 16th Edition, 1995), but with some modifications according to the EPA.
3. Clinical hand sanitizer study: for final evaluation of either wet or dry product (following the ASTM E1174 standard or a modification thereof)

In the United States, the FDA's OTC (over the counter drug products) division is responsible for regulating antiseptic handwash products. The FDA requires a clinical study in which the hands of volunteers are inoculated with 5 mL of a standardized suspension of a bacterium, Serratia marcescens. Per ASTM standard E1174, ten washes with a test formulation are required. The FDA requires, per its tentative final monograph from 1994, that there is a 2-log₁₀ reduction of organisms on each hand within 5 minutes after the first use and a 3-log₁₀ reduction on each hand within 5 minutes after the tenth use (per FDA's tentative final monograph of 1994).

All publications, patents, and patent documents cited herein are incorporated by reference herein, as though individually incorporated by reference. The invention has been described with reference to various specific embodiments and techniques. However, it should be understood that many variations and modifications can be made while remaining within the spirit and scope of the invention.

What is claimed is:

1. A topical adhesive patch comprising:
a backing having a front side and a back side;
at least one antimicrobial that is integrally bonded to the back side of the backing; and
a formulation in contact with the front side of the backing, the formulation comprising an adhesive.

2. The adhesive patch of claim 1 wherein the formulation is positioned on at least a portion on the front side of the backing, in at least a portion on the front side of the backing, or on and in at least a portion on the front side of the backing.

3. The adhesive patch of claim 1, wherein the backing is flexible.

4. The adhesive patch of claim 1, wherein the antimicrobial is integrally bonded to fibers of the backing, such that the antimicrobial is incorporated into the fibers.

5. The adhesive patch of claim 1, wherein the antimicrobial is integrally bonded to the surface of the back side of the backing.

6. The adhesive patch of claim 1, wherein the antimicrobial is chemically bonded to the backing.

7. The adhesive patch of claim 1, wherein the antimicrobial comprises a quaternary ammonium chloride, or an alkylammonium cation.

8. The adhesive patch of claim 7, wherein the antimicrobial comprises 3-(trimethoxysilyl)propyl dimethyl octadecyl ammonium chloride.

9. The adhesive patch of claim 1, wherein the antimicrobial comprises sialic acid.

10. The adhesive patch of claim 1, wherein the backing of the patch is porous.

11. The adhesive patch of claim 1, wherein the backing of the patch is vapor permeable.

12. The adhesive patch of claim 1, wherein the backing of the patch comprises a non-woven fabric.

13. The adhesive patch of claim 1, wherein the backing of the patch comprises at least one of polycellulose fibers, polyester fibers, polyurethane fibers, polyolefin fibers, polyamide fibers, cotton fibers, copolyester fibers, and films.

14. The adhesive patch of claim 1, wherein upon contact with skin, the backing of the patch retains the formulation and the adhesive patch allows moisture from the skin to pass through the adhesive patch.

15. The adhesive patch of claim 1, wherein the adhesive comprises at least one of an acrylic ester copolymer, a water-based adhesive, a hot melt adhesive, a pressure sensitive adhesive, a solvent based pressure sensitive adhesive, a polyacrylate, a polyisobutylene, a polybutene, a rubber, a silicone based pressure sensitive adhesive, a polystyrene-polybutadiene-polystyrene block polymer, a polystyrene-polyisoprene-polystyrene block polymer, and a polystyrene-poly(ethylene-butylene)-polystyrene block polymer.

16. The adhesive patch of claim 1 that is safe and non-toxic upon accidental ingestion.

17. The adhesive patch of claim 1 that causes minimal or no skin irritation upon use.

18. The adhesive patch of claim 1 that causes little or no irritation upon contact with an eye, or surrounding tissue thereof.

19. A method of reducing the number of microbes located upon a topical skin surface of a mammal, the method comprising topically contacting a skin surface of the mammal with the back side of a topical adhesive patch comprising:
a backing having a front side and a back side;
at least one antimicrobial that is integrally bonded to the back side of the backing; and
a formulation in contact with the front side of the backing, the formulation comprising an adhesive;
effective to reduce the number of microbes located upon a topical skin surface.

20. A method of preventing the transmission of a communicable disease capable of being transmitted by physical contact, the method comprising topically contacting a skin surface of the mammal at risk of such communicable disease, with the back side of a topical adhesive patch comprising:
a backing having a front side and a back side;
at least one antimicrobial that is integrally bonded to the back side of the backing; and
a formulation in contact with the front side of the backing, the formulation comprising an adhesive;
effective to prevent the transmission of the communicable disease.

21. The method of claim 20 wherein the communicable disease comprises at least one of the following: common cold (rhinovirus), human Influenza (Influenzavirus A, Influenzavirus B, Influenzavirus C or Influenza H1N1), staphylococcus infection, streptococcus infection, gastroenteritis, bacterial meningitis, conjunctivitis, bacterial pneumonia, pertussis, tonsillitis, infectious diarrhea, cellulitis, impetigo, folliculitis, scalded skin syndrome, urinary tract infection, ringworm, athlete's foot, yeast infection, bronchiolitis, croup, measles, mumps, rubella, infectious diarrhea, encephalitis, conjunctivitis, chicken pox, west Nile virus, mononucleosis, cold sores, and Avian influenza A (H5N1) virus.

22. The method of any one of claims 20, wherein the mammal is a human that is younger than 18 years of age.

23. The method of claim 20, wherein the topically contacting the skin surface of the mammal with the back side of the topical adhesive patch comprises rubbing the front side of a hand with the back side of the topical adhesive patch.

24. The method of claim 20, wherein the topical adhesive patch mechanically traps, removes, inhibits, or kills harmful pathogens from the skin surface of the mammal

25. The method of claim 20, wherein the topical adhesive patch chemically traps, removes, inhibits, or kills harmful pathogens from the skin surface of the mammal

26. The method of claim 20, wherein less than about 1 wt. % of the antimicrobial from the topical adhesive patch is dispensed or transferred to the skin surface of the mammal.

27. The method of claim 20, wherein the topical adhesive patch can remain active and effectively prevent the transmission of the disease for at least about 8 hours.
